(19) United States
(12) Patent Application Publication
(10) Pub. No.: US 2009/0258368 A1
(43) Pub. Date: Oct. 15, 2009

(54) PARAMAGNETIC NUCLEATION NANOPARTICLES

(76) Inventors: John M. Noonan, Rochester, NY (US); Charles DeBoer, Ithaca, NY (US); Richard S. Murante, Rochester, NY (US); David B. Bailey, Webster, NY (US); D. Michael Connolly, Rochester, NY (US)

Correspondence Address: HISCOCK & BARCLAY, LLP, 2000 HSBC PLAZA, 100 Chestnut Street, ROCHESTER, NY, 14604-2404, US

(21) Appl. No.: 12/423,288

(22) Filed: Apr. 14, 2009

Related U.S. Application Data
(60) Provisional application No. 61/044,840, filed on Apr. 14, 2008.

(51) Int. Cl. C12Q 1/68

(52) U.S. Cl. 435/6

(57) ABSTRACT

A nucleic acid binding substance which has affinity for nucleic acid polymers. The nucleic acid binding substance is comprised of a nucleic acid binding element capable of specific binding to nucleic acid molecules and connected to a nucleation nanoparticle having paramagnetic properties.

### CROSS-REFERENCE TO RELATED APPLICATIONS

This application claims priority from U.S. Provisional Patent Application Ser. No. 61/044,840, filed Apr. 14, 2008.

### FIELD OF THE INVENTION

This invention relates to a nucleic acid binding substance containing paramagnetic nucleation nanoparticles. In another embodiment the invention relates to a nucleic acid binding substance containing nucleation nanoparticles having paramagnetic and catalytic properties.

### BACKGROUND OF THE INVENTION

In running biological and chemical tests it is often desired to concentrate a sample to retain desired analyte. Concentrating the sample can be a difficult process. Traditional methods for concentrating a biological sample include filtering, rinsing, centrifuging and/or reaction chemistry. Often these steps cannot be preformed in a single processing chamber and require the sample to be transferred to other devices or chambers.

Paramagnetic particles are particles which are attracted to a magnetic field. Unlike ferromagnetic particles, paramagnetic particles retain little or no magnetic properties in the absence of a magnetic field. By attaching a paramagnetic nucleation particle to nucleic acid polymers and applying a magnetic field to a sample, the nucleic acid polymers can be moved to a desired location, thereby concentrating a portion of the sample with the nucleic acid polymers. The sample can then be drawn from the concentrated portion yielding a high amount of nucleic acid polymers.

Applying a magnetic field further allows for manipulating the nucleic acid polymer into distinct chambers at speeds faster than the diffusion rate. Additionally, by holding a nucleic acid polymer steady a rinse can be applied without washing away the nucleic acid polymer.

Therefore, a nucleation particle that specifically binds to target nucleic acid molecules is desired.

Further, a nucleation particle having paramagnetic properties is desired.

Nucleic acid molecules by themselves are not sufficiently electrically conductive to be useful for electronic circuits. Therefore, in applications where it is desired to use nucleic acid molecules as a conductor, metal is deposited onto the nucleic acid molecule to form an electrically conductive molecule.

U.S. Pat. No. 6,399,303 issued to Connolly on Jun. 4, 2002, which is hereby incorporated by reference, discloses a method and device for detecting target nucleic acid molecules across a set of oligonucleotide probes integrated into an electric circuit. A target nucleic acid molecule is coated with metal to electrically detect the target nucleic acid molecule. The metal specifically coats the target nucleic acid molecule while not coating other non-target sites to avoid shorting out the system. Additionally, the metal coating process does not interfere with the hybridization of the target nucleic acid molecule.

To effectively coat a target nucleic acid with metal, first, a catalytic metal compound is attached to the target nucleic acid. Next, the target nucleic acid molecule is treated with a developer solution containing a reducing agent and metal ions. The nucleic acid catalytic metal compound promotes the reduction of the metal ions to metal. The metal ion reduction proceeds to coat the target nucleic acid molecule with metal. Connolly teaches catalytic metals attached to the target in the form of single atoms. To speed the deposition reaction of metal onto the target nucleic acid molecule a catalytic nucleation particle containing a larger amount of catalyst metal is desired.

One method to increase the concentration of the catalyst is through the use of a nucleation nanoparticle having a high concentration of catalytic groups. However, particles, and molecules in general, have been known to non-specifically bind to molecules and surfaces other than the target nucleic acid molecule. Non-specific binding of nucleation nanoparticles leads to metal coating in undesirable areas. In electrical detection devices the metal coating has the potential to create shorts that result in false positives. To reduce the non-specific binding of particles to non-desired surfaces stabilizers are added to the particle surface. The addition of stabilizers limits the non-specific binding of the particle, however, these particles have no inherent preference for binding to nucleic acid polymers. Therefore, it is desirable to functionalize the nucleation nanoparticle with a binding compound or combination of compounds having a specific preference for binding the target nucleic acid polymer or a binding group attached thereon.

Therefore, a nucleation particle that has a high concentration of catalytic molecules to provide a rapid reaction time is desired.

Further, a nucleation particle that causes minimal background disruptions and no adverse effects on hybridization is desired.

### SUMMARY OF THE INVENTION

The invention comprises, in one form thereof, a paramagnetic nucleation nanoparticle containing a nucleic acid binding element to bind the nucleation nanoparticle to nucleic acid polymers. In its simplest form, the nucleic acid binding element links directly to the particle surface. Optionally, the nucleic acid binding element is attached to the paramagnetic nucleation nanoparticle via intermediate connecting groups such as, but not limited to linkers, scaffolds, stabilizers or steric stabilizers. The intermediate connecting group can be of variable size, architecture and chemical composition to interconnect the paramagnetic nucleation nanoparticle(s) and the nucleic acid binding element(s) into a multifunctional entity. In another embodiment, the paramagnetic nucleation nanoparticle further contains a catalytic material.

In one embodiment, the nucleic acid binding group functionalized particle requires improved colloid stability to prevent agglomeration. Therefore, a colloid stabilizer, such as a hydrophilic chain or ionic group, is added or connected to a linking group that links to the particle. These groups assist in limiting the nanoparticles size during the particle generation stage.

In another form, the invention includes a method for specifically binding a catalytic nucleation nanoparticle to a target nucleic acid molecule.

An advantage of the present invention is that the utilization of paramagnetic nucleation nanoparticles allows for sample concentration by applying a magnetic field without additional processing steps.

A further advantage of the present invention is that the utilization of catalytic nucleation nanoparticles enhances specific metallization of the nucleic acid molecule and reduces reaction time.

### BRIEF DESCRIPTION OF THE DRAWINGS

The present invention is disclosed with reference to the accompanying drawings, wherein:

FIG. 1 shows two embodiments of the present invention, where the dashed outline indicates an optional scaffold component;

FIG. 2 is a graphical representation of a nucleation nanoparticle attached to a nucleic acid polymer;

FIG. 3 shows the hybridized sensor test method used to evaluate the materials includes building a microelectronic array of conductive wires; and

FIG. 4 is an SEM image of metal wires along DNA connecting gold wires.

Corresponding reference characters indicate corresponding parts throughout the several views. The examples set out herein illustrate several embodiments of the invention but should not be construed as limiting the scope of the invention in any manner.

### DETAILED DESCRIPTION

The nucleic acid binding substance has affinity for target nucleic acid polymers. The nucleic acid binding substance contains a nucleic acid binding element capable of specific binding to target nucleic acid polymers and is attached to a paramagnetic nucleation particle. Optionally, the nucleic acid binding element is attached to the paramagnetic nucleation nanoparticle via at least one intermediate connecting group such as, but not limited to linkers, scaffolds, stabilizers or steric stabilizers.

The nucleation nanoparticle contains particles that exhibit paramagnet properties. There are a number of particles that exhibit paramagnetic properties. In one embodiment, cobalt, nickel, iron or a combination thereof is used to create a paramagnetic nucleation nanoparticle. Optionally, the paramagnetic nucleation nanoparticle further contains a catalytic particle. In one embodiment, the catalytic particle is palladium, platinum, silver or gold.

In another embodiment, the nucleation nanoparticles contain ferrimagnetic particles. The ferrimagnetic particles include metal oxides, such as iron oxides. Suitable iron oxides include hematite (Fe₂O₃) and magnetite (Fe₃O₄).

In one form, a nickel-palladium nanoparticle, stabilized by a surface layer of 4-dimethylaminopyridine as described in Flanagan et al, Langmuir, 2007, 23, 12508-12520, is treated by adsorption with a plurality of ethidium bromide intercalator molecules to create nucleic acid binding sites. The ethidium moiety bonds to the nucleic acid polymer, thereby attaching the nickel-palladium nanoparticle to the nucleic acid polymer.

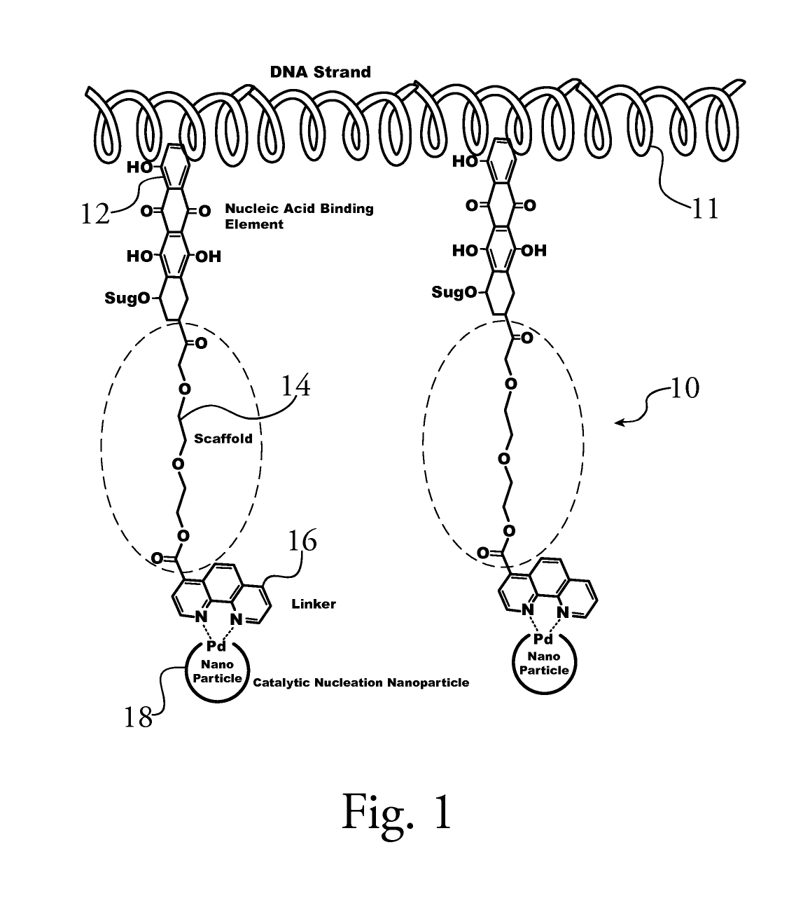

In another form, as illustrated in FIG. 1, a simple straight-chain scaffold molecule 14, such as oligoethylene glycol (PEG), is affixed with a nucleic acid binding element 12 at one end and a linker 16 at the other end. The nucleic acid binding element 12 binds to the nucleic acid polymer 11 and the linker 16 binds to the catalytic nucleation nanoparticle 18. In one embodiment, the nucleic acid binding element 12 is an intercalator, such as ethidium bromide, or a minor groove binder such as distamycin. The linker is a phenanthroline derivative. Hainfeld, J. Structural Biology, 127, 177-184 (1999) reports the advantage of phenanthroline derivatives in creating palladium particles. The scaffold 14 may be a simple difunctional straight chain as shown, or may be a multifunctional branched scaffold connecting multiple catalytic nucleation nanoparticles or nucleic acid binding elements. The nucleic acid binding element bonds to the nucleic acid polymer, thereby attaching the palladium nanoparticle to the nucleic acid polymer. It is understood that additional nucleic acid binding elements and intermediate connecting groups are within the scope and may be used.

Referring again to FIG. 1, the nucleic acid binding substance 10 has a nucleic acid binding element 12, such as an intercalator or minor groove binder, a catalytic nucleation particle 18, such as a palladium nanocluster, and optionally, a linker 16, which serves as the linking site for the nucleation nanoparticle 18. It is noted that in this embodiment, the phenanthroline linker also serves as a stabilizer to control particle growth and stability. Optionally, a scaffold composition, such as the straight chain linker as shown, is used to attach the nucleic acid binding element to the linker.

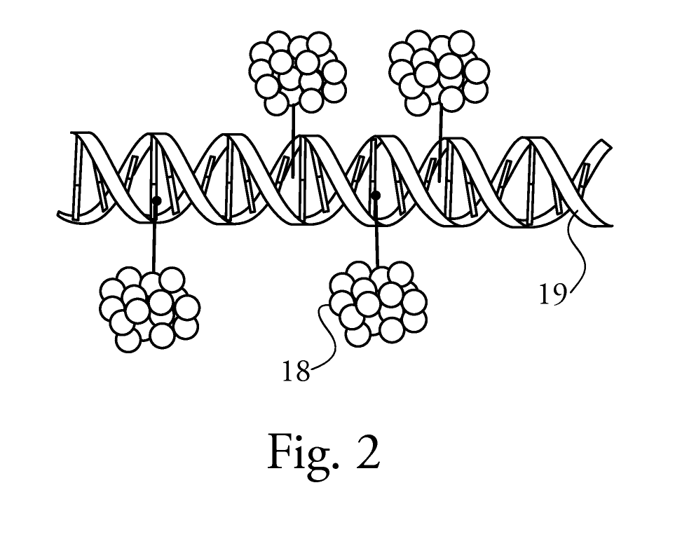

Referring to FIG. 2, there is shown a graphical representation of a catalytic nucleation particle 18 attached to a nucleic acid polymer 19.

It is understood that the terms “attachment” and “in communication with” as used herein are not limited to binding one element directly to another and that an intermediate connecting group may be utilized to facilitate attachment. The term “binds” or “bound” refers to a direct attachment with no intermediate connecting group.

### Paramagnetic Nucleation Nanoparticles:

Use of sols or clusters in the form of paramagnetic nucleation nanoparticles allows for the attachment of paramagnetic material to a target nucleic acid polymer or other target analyte. By applying a magnetic field to the sample, the nucleic acid polymer can be manipulated via the attached paramagnet material.

The paramagnet nucleation nanoparticles are formed in solution with a stabilizer. In one embodiment, a metal salt is used. A reducing agent, such as dimethylamineborane or sodium borohydride, is added to the solution. If needed, solvents and excess salts can be removed by centrifugation, decantation, washing, and resuspension of the metal clusters. Alternatively, a magnetic field can be applied to the solution holding the paramagnetic nucleation nanoparticles in place as a drain and rinse is applied.

### Paramagnetic Nucleation Nanoparticles Containing Catalytic Particles:

Use of sols or clusters in the form of paramagnetic nucleation nanoparticles containing catalytic particles improves the metallization of the target nucleic molecule by attaching a large amount of catalytic material to the target nucleic acid. A paramagnetic nucleation nanoparticle containing multiple catalytic agents can be attached to a single binding site. A sol is a suspension of metal or metal precursor particles. In one embodiment, the paramagnetic nucleation nanoparticles contain a catalytic metal such as palladium, platinum, silver, gold, palladium alloy, platinum alloy, silver alloy, gold alloy or a combination thereof. In another embodiment, the nucleation nanoparticles contain catalytic precursors in the form of ions. (see W. L Dressick, L. M. Kondracki, M. Chen, S. L Brandow, E. Matijevic, J. Calvert, Colloids and Surfaces, 108, 101-111, 1966). The ions are reduced to metal in the final metallization treatment. In another embodiment, the paramagnetic nucleation nanoparticles contain a mixture of metal precursors in the form of metal ions.

### Nucleic Acid Binding Element:

The nucleic acid binding element attaches to the paramagnetic nucleation nanoparticle, either directly or by way of an intermediate connecting group. The nucleic acid binding element further binds to the nucleic acid polymer. In one embodiment, the nucleic acid polymer spans an electrical gap, thereby enabling an electrical connection across the gap. The nucleic acid polymer itself does not provide sufficient conductive properties to complete the circuit. The nucleic acid binding element is any molecule, fragment or functional group that binds to nucleic acid polymers. Potential nucleic acid binding elements consist of intercalators, minor groove binders, cations, amine reactive groups such as aldehydes and alkylating agents, proteins, and association with hydrophobic groups of surfactants. In addition, functional groups such as aldehydes are used to create a connection by reaction with free amines in the nucleic acid. Other amine reactive groups, such as Michael addition, are suitable.

Examples of structures that form the basis for intercalating and minor groove binder structures are:

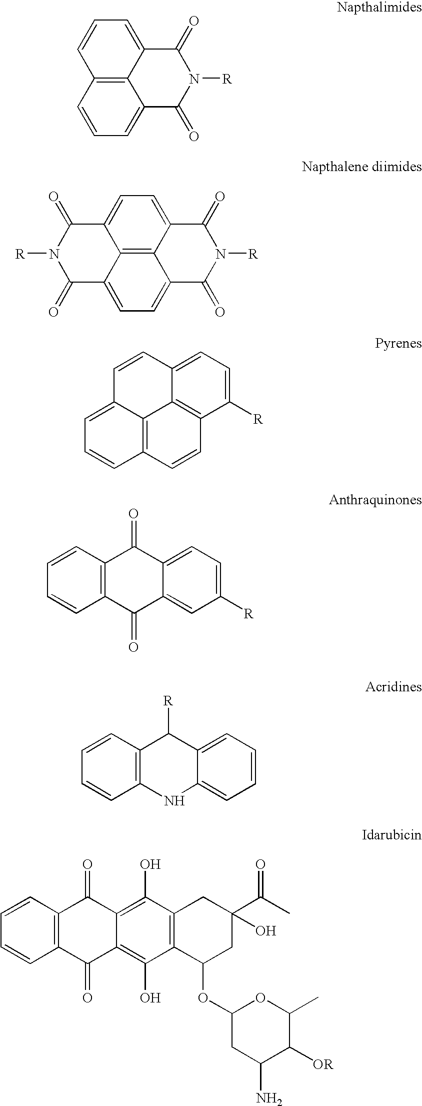

The range of specific intercalator and minor groove binder structures is enormous as the field has been the subject of intense study for over 50 years. See R. Martinez and L Chacon-Garcia, Current Medicinal Chemistry, 2005, 12, 127-151. Therefore, the R groups include a broad range of organic functional groups. In many cases, interaction can be enhanced if R contains hydrogen bonding, cationic or hydrophilic character.

In addition, compounds such as cationic polymers, such as polyethyleneimine, interact with nucleic acid and have been proposed as gene carriers as evidenced by Xu et al., International Journal of Nanoscience, 2006, 5, 753-756 and Petersen et al., Bioconjugate Chemistry, 2002, 13, 845-854. Proteins are another well known class of materials that offer useful nucleic acid interaction and could be the basis for attaching nanoparticles to nucleic acids. Direct reaction with functional groups on the nucleic acid is also within the scope of this invention. For example, amine groups can be reacted with aldehydes to create a bond (Braun et al, Nano Letters, 2004, 4, 323-326)

In one embodiment, the nucleic acid binding elements are specific binding agents that specifically target double-stranded nucleic acid molecules while not binding with single-stranded nucleic acid molecules. For example, minor-groove binding compounds specifically bind hybridized double-stranded DNA molecules, but do not bind to single-stranded oligonucleotide capture probes. In contrast, palladium chloride reagent indiscriminately binds to both the target molecules and capture probes. The binding element binds specifically to the target nucleic acid molecule while having little or no affinity towards non-target molecules. It is understood that the specific binding elements can include, but are not limited to, intercalators, minor-groove binding compounds, major-groove binding compounds, antibodies, and DNA binding proteins. The specific binding element binds to a specific site on a target nucleic acid without binding to non-desired areas. In one embodiment, the specific binding element is ethidium bromide. In alternative embodiments, the specific binding element is distamycin, idarubicin, or Hoescht dye.

In one embodiment, the nucleic acid binding element also serves as a stabilizer as described below.

### Stabilizers:

The paramagnetic nucleation nanoparticles are surface functionalized with stabilizers to impart desirable properties. These stabilized nucleation nanoparticles demonstrate colloid stability and minimal non-specific binding. Furthermore, the presence of the stabilizer in solution while forming the paramagnetic nucleation nanoparticle controls the nanoparticle size.

The stabilizer provides colloid stability and prevents coagulation and settling of the paramagnetic nucleation nanoparticle. The stabilizer further serves to limit the size of the paramagnetic nucleation nanoparticles during the formation process. In one embodiment, metal paramagnetic nucleation nanoparticle are formed in a solution containing stabilizer and metal ions. In one embodiment, the stabilizers are chelating compounds. Large paramagnetic nucleation nanoparticles are undesirable as they are more likely to precipitate out of solution. Therefore, the nucleation nanoparticle shall be small enough to remain in solution. In one embodiment, the nucleation nanoparticle is generally spherical in shape with a diameter from about 0.5-1000 nm. Preferably, the nucleation nanoparticle is generally spherical in shape and has a diameter from about 1-100 nm.

Suitable stabilizers include, but are not limited to, polyethyloxazoline, polyvinylpyrollidinone, polyethyleneimine, polyvinylalcohol, polyethyleneglycol, polyester ionomers, silicone ionic polymers, ionic polymers, copolymers, starches, gum Arabic, suractants, nonionic surfactants, ionic surfactants, fluorocarbon containing surfactants and sugars. In one embodiment, the stabilizer is a phenanthroline, bipyridine and oligovinylpyridine of the following formulas:

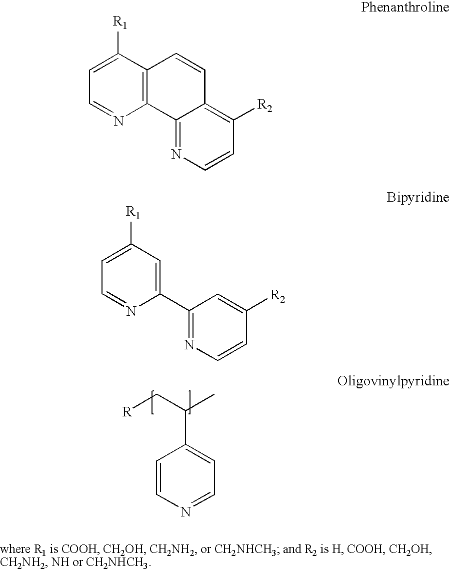

In one embodiment where the paramagnetic nucleation nanoparticle contains palladium, these stabilizers link by acting as ligands for palladium ions and are therefore closely associated with the particle formation. In addition to linking, the stabilizers have hydrophilic groups that interact with the water phase. The linking and stabilization function of molecules such as phenathrolines in palladium particle formation is further described in Hainfeld, J. Structural Biology, 127, 177-184 (1999).

It is understood that particles derived from a broad class of materials (plastics, pigments, oils, etc) in water can be stabilized by a wide array of surfactants and dispersants that do not rely on specific coordination. These classes of stabilizers are also within the scope of this invention.

In one embodiment, the stabilizer stabilizes the paramagnetic nucleation nanoparticle from precipitation, coagulation and minimizes the non-specific binding to random surfaces. In another embodiment, the stabilizer further functions as a nucleic acid binding element as described below.

### Linker:

The linker is bound directly to the paramagnetic nucleation particle to allow the attachment of other intermediate connecting groups or nucleic acid binding elements. It is understood that the linker can also serve as a stabilizer or scaffold.

The linker can be bound through various binding energies. The total binding energy consists of the sum of all the covalent, ionic, entropic, Van der Walls and any other forces binding the linker to the catalytic nucleation nanoparticle. In one embodiment, the total binding energy between the linker and the paramagnetic nucleation particle is greater than about 10 kJ/mole. In another embodiment, the total binding energy between the linker and the paramagnetic nucleation particle is greater than about 40 kJ/mole. Suitable linkers include, but are not limited to, ligands, phenanthrolines, bidentates, tridentates, bipyridines, pyridines, tripyridines, polyvinylpyridines, porphyrins, disulfides, amine acetoacetates, amines, thiols, acids, alcohols and hydrophobic groups.

### Scaffold Compositions:

The paramagnetic acid binding element may be connected directly to the catalytic nucleation particle or a linker. Alternatively, the nucleic acid binding element is attached to a scaffold, either individually or as a multiplicity. In either case, the final conjugate is endowed with the two essential properties—nucleic acid specific recognition-binding and an attached paramagnetic nucleation nanoparticle. Attaching the nucleic acid binding element to the scaffold may be by way of any of the common organic bonding groups such as esters, amides and the like.

Referring again to FIG. 1, the nucleation nanoparticle is attached via a linker, such as a ligand, which is in turn connected to the nucleic acid binding element through an optional scaffold. The scaffold creates a greater space between the nucleation nanoparticle and the nucleic acid molecule. In a simple case, this can be achieved with a spacer or scaffold that is a linear bifunctional oligomer. Higher order structures may also be obtained with multi functional oligomer, polymer, dendrimer, microgel or latex particles. The enhanced spacing afforded by the scaffold composition reduces physical interference between the particle and the nucleic acid polymer. This allows for the use of larger nucleation nanoparticles without interfering with binding of the nucleic acid binding group to the nucleic acid molecule. The linker can be any compound that does not substantially interfere with nucleic acid molecule interactionthe oligonucleotide probes.

Attachment to a common scaffold creates an enormous range of possible sizes, shapes, architectures and additional functions. In one embodiment, the scaffold composition is a linear chain with the two functional groups at the ends. The chain itself can be of any composition, length and ionic character. In an alternative embodiment, often used in biological applications, polyethylene glycol with a reactive amine, acid or alcohol end groups is utilized as included in the following example:

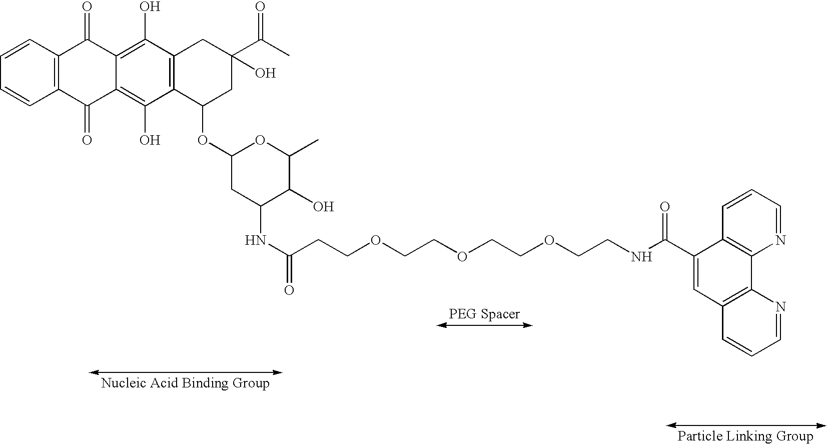

Linear short spacers with cationic character can be desirable as they can enhance intercalation performance.

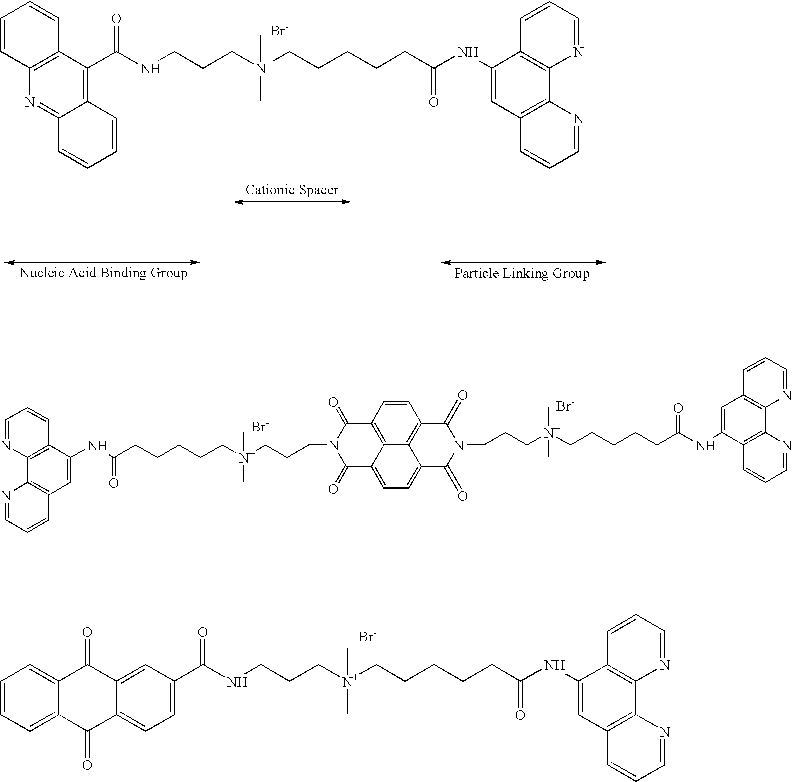

A polymeric or oligomeric scaffold allows for multiple groups to be joined in the same structure where the number of groups is limited only by the size of the chain.

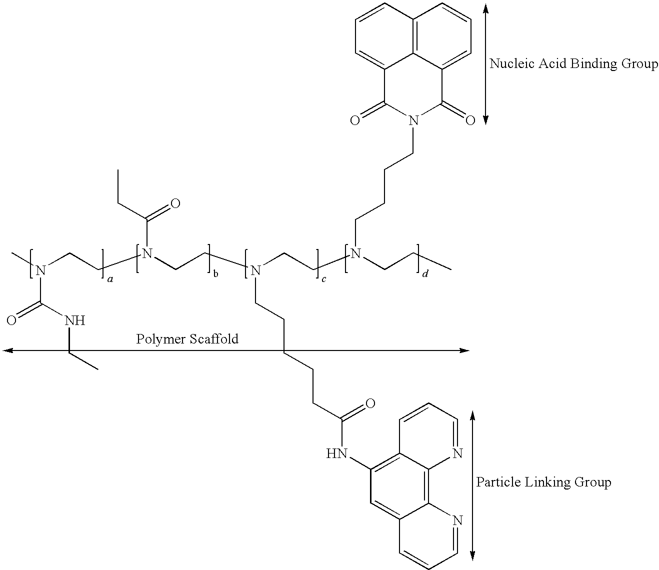

In addition to short and long chain structures. scaffolds can be built with branched or very highly branched architectures. Furthermore, scaffolds can be a microgel particle with nanoparticles bound to a swollen polyvinylpyridine interior and peripheral nucleic acid binding elements are illustrated. In another embodiment. the scaffold is a core-shell latex particle with nucleation nanoparticles centers and peripheral nucleic acid recognition groups populating the surface. It is understood that any scaffold compositions can be incorporated to connect intermediate connecting groups, catalytic nucleation nanoparticles or nucleic acid binding elements.

### Steric Stabilizers:

In one embodiment, a steric stabilizer is used to attach the nucleic acid binding element to the paramagnetic nucleation nanoparticle. The steric stabilizer is capable of functioning as a stabilizer, linker and scaffold as described above. In one embodiment, the steric stabilizer is polyethylenimine, polyethyloxazoline or polyvinylpyrrolidone. The steric stabilizer binds to the paramagnetic nucleation particle with a total binding energy of at least 10 kJ/mole. In another embodiment, the steric stabilizer binds to the paramagnetic nucleation particle with a total binding energy of at least 40 kJ/mole. The use of steric stabilizers eliminate any need for distinct stabilizers, linkers, or scaffolds. One or multiple nucleic acid binding elements can be attached to the steric stabilizer. Furthermore, one or multiple paramagnetic nucleation nanoparticles can be bound to the steric stabilizer.

### Nucleic Acid Binding Substance:

In one embodiment for forming the nucleic acid binding substance on a nucleation nanoparticle, the nucleation nanoparticles are formed in solution with a stabilizer such as dimethyaminopyridine (DMAP). The stabilized nucleation nanoparticles are purified to retain clusters of the desired size. The nanoparticles are then treated directly with a nucleic acid binding element such as ethidium bromide or with a nucleic acid binding element connected to a linker or with a scaffold composition containing the nucleic acid binding element. The scaffold composition can be a polymer containing nucleic acid binding elements such as napthalimide or acridine. The polymer displaces some of the DMAP and attaches to the particle. It is understood that the nucleic acid binding element can be chemically attached to the scaffold composition prior to the attachment of the scaffold composition to the particle.

In another embodiment for forming the nucleic acid binding substance on a nucleation particle, the nucleation nanoparticles are formed in solution in the presence of a nucleic acid binding element such as ethidium bromide or in the presence of a nucleic acid binding element connected to a linker or in the presence of a scaffold composition containing the nucleic acid binding element. The scaffold composition can be a polymer containing nucleic acid binding elements such as napthalimide or acridine. It is understood that the nucleic acid binding substance connects to the particle during the particle formation process and may offer some colloidal stability to the dispersion. In addition, stabilizers in the form of ionic surfactants, non ionic surfactants, water soluble oligomers and polymers may also be added to enhance colloid stability and control particle size.

### Paramagnetic Examples:

Metal salts (nickel, cobalt, iron) with a small amount of palladium salt are dissolved in a solvent (water and/or polar organic solvent) along with a stabilizer (phenanthroline, bipyridine, polyvinylpyrrolidinone). A reducing agent is added (dimethylamineborane, sodium borohydride) and the mixture is held until the metal clusters are formed. If needed, solvents and excess salts can be removed by centrifugation, decantation, washing, and resuspension of the metal clusters.

Solution A—24 g of nickel chloride hexahydrate and 44 g of sodium citrate were dissolved in 500 ml of water.

Solution B—24 g of ethanolamine were dissolved in 500 ml of water.

Solution C—5 g of cobalt chloride hexahydrate were dissolved in 100 ml water.

Solution D—2 g of potassium tetrachloropalladate and 6 g of potassium chloride were dissolved in 100 ml of water.

Solution E—1 g of bathophenanthroline-disulfonic acid, disodium salt hydrate was dissolved in 100 ml water.

Solution F—3 g of dimethylamine borane were dissolved in 100 ml water.

### EXAMPLE 1

In a 20 ml glass vial, 1 ml solution A and 1 ml of solution B were mixed. 0.1 ml of solution D was added, followed immediately by 0.2 ml of solution E. Then 0.5 ml of solution F was added and the mixture was held at 60 degrees C. for 30 minutes. After cooling to room temperature, the mixture was placed in a strong magnetic field for 10 seconds (the magnetic field was from the permanent magnetic removed from a discarded computer hard drive) and it was observed that most of the metal clusters moved to the wall of the vial nearest the magnet.

### EXAMPLE 2

In a 20 ml glass vial, 0.2 ml solution A, 0.8 ml solution C and 1 ml of solution B were mixed. 0.1 ml of solution D was added, followed immediately by 0.2 ml of solution E. Then 0.5 ml of solution F was added and the mixture was held at 60 degrees C. for 30 minutes. After cooling to room temperature, the mixture was placed in a strong magnetic field for 10 seconds (the magnetic field was from the permanent magnetic removed from a discarded computer hard drive) and it was observed that most of the metal clusters moved to the wall of the vial nearest the magnet.

### Metallic Coating Examples:

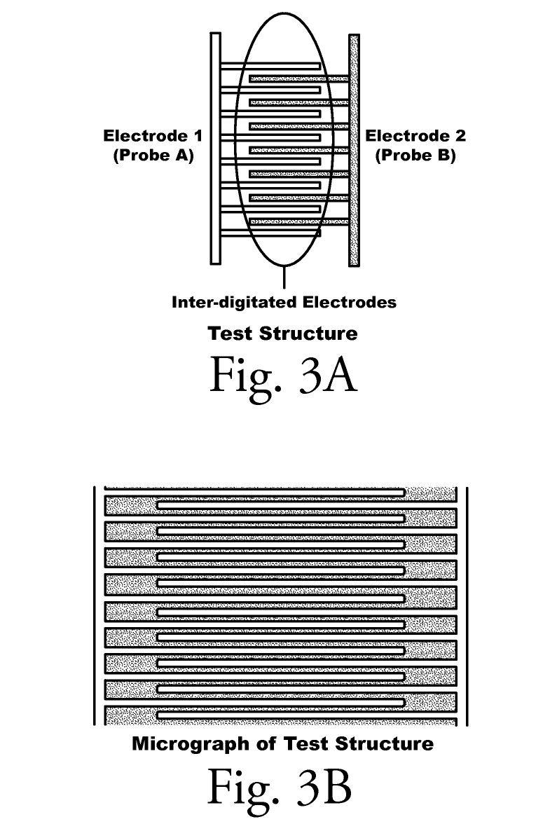

Referring to FIG. 3, the hybridized sensor test method used to evaluate the materials includes building a microelectronic array of conductive wires, such as 2 μm gold wires and insulative spacers, such as 1 μm silicon oxide spaces, attaching nucleic acid oligomer probes to the gold, binding a nucleic acid polymer across the gold wires to the probes, treating the array with catalytic nucleation nanoparticles and then development in a conductive bath, such as a nickel bath.

The sensor chips consist of arrays of paired electrodes connected to electrical contact pads. Standard microelectronic techniques are used for the fabrication of the sensor chips. These processing methods include thermal oxidation, thermal evaporation, photolithography, chemical etching, standard metrology, and electrical testing. The starting substrate is a P-type 6-inch silicon wafer with <100> orientation and resistivity less than 1 Ohm-cm. Electrodes are fabricated by evaporating thin films of gold.

Oligonucleotide probes were purchased from TriLink Biotech. Synthesis of these oligonucleotides included 3′-modifications of a C6-thiol, required for sulfur-specific binding to gold, and a polyethylene glycol (PEG) spacer of 18 chain atoms for enhanced hybridization efficiency. The sequences of the probes made to the lef and pagA genes were 5′HS-C6-X-GAGCGATTCTT TATCTGAAGAAGGAAG3′ and 5′HS-C6-X-GAGTGAAGTGTTACCGCAAATTCAAGA A3′, where HS indicates the thiol group, C6 is a chain of six aliphatic carbon atoms, and X indicates the PEG18 spacer.

DNA containing the complimentary base sequence for binding to the probes was diluted to a concentration of 200, 500 or 1000 ng/60 μl in hybridization buffer (60 mM Na citrate (pH 7.5), 1.5% dextran sulfate, and 0.1% SDS). The resulting DNA solutions were heated for 5 minutes at 95° C. to denature the DNA. Microchips were held individually in custom plastic trays made to fit inside 10×10 cm Petri plates. Two milliliters of hybridization buffer was added in the Petri dishes under the tray to maintain a humid environment when incubations were performed. The sensor chips were pre-incubated under these conditions for 10 minutes at 55° C. Sixty microliters of the denatured DNA solution were applied to cover the entire surface of the pre-incubated sensor chips to provide the specified amount of DNA. Hybridization reactions were carried out for 20 minutes in Petri dishes and incubated on the surface of an aluminum heat block set at 55° C. At the end of the hybridization, 540 μl of buffer was added and the microchips were transferred to a Petri dish for subsequent washing steps. The post hybridization washes consisted of two rinses with 20 ml hybridization buffer and two additional rinses with 20 ml of 10 mM Na citrate (pH 7.6) solution. Immediately after the second 10 mM citrate rinse, additional 10 mM citrate buffer was poured into the dish so that the sensor chips remain covered with liquid. Individual sensor chips were taken out of the citrate buffer, dipped into a beaker containing 50 ml of ddH2O, and then dried in a stream of N2. A second stage of rinsing consisted of four rinses with 20 ml ddH2O. After the final rinse, each sensor was individually removed from ddH2O and dipped into a fresh 50 ml of ddH2O, and then dried with N2.

The test chip was treated with an aqueous dispersion of the nucleation particle composition of interest for a specified period of time, rinsed with water and then immersed in a nickel plating bath consisting of 0.43% nickel chloride, 1.28% sodium citrate, 0.8% ethanolamine, and 2% dimethylaminoborane. The metallization process was followed either by microscopic inspection for the presence of wire formation or by electrical testing of resisitivity versus time. A precipitous drop in resistivity indicated electrical connection between the gold wires.

The electrical test uses a Kelvin measurement technique to determine the resistance of the test structure. The Current Source (CS) is composed of two circuits: a Current Stimulus Circuit (CSC) and a Current Measurement Circuit (CMC). The CSC applies a voltage potential across the test structure and the CMC measures the resulting current. At the same time, the Voltage Measurement Circuit (VMC) measures the applied voltage.

In order to measure a single test structure, the CSC applies an initial voltage of 1 mV across the test structure in question. A reading, consisting of sixteen (16) voltage and current measurements that are averaged to minimize the effects of noise, is made and compared to set voltage (5 mV) and current (5 μA) limits. Each microchip contains fourteen (14) test structures. They are measured serially (structure #1 first, structure #2 second, . . . and structure #14 fourteenth) using four (4) multiplexers to switch the positive and negative VMC terminals and the in and out terminals for the CS. Resistance is determined using Ohm's law. The logarithm (base 10) of the resistance measurement data for each feature is averaged across the fourteen features to give an average log10 resistance for the sensor (AvelogR).

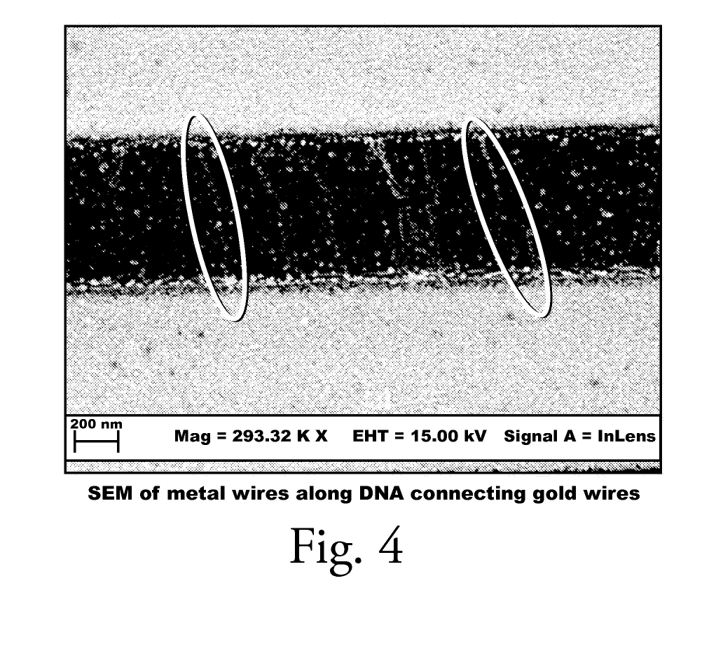

Referring to FIG. 4, there is shown an SEM of metal wires, along the DNA strands, connecting the gold wires similar to those of the microelectronic array shown in FIG. 3.

### EXAMPLE 3

A palladium nanoparticle functionalized and stabilized with dimethylaminopyridine (DMAP) was made as follows: Potassium tetrachloropalladate (II) (41.5 mg, 0.127 mmol) was dissolved in distilled-deionized water (3 ml) and stirred vigorously. 4-Dimethylaminopyridine (DMAP) (83.3 mg, 0.682 mmol) was dissolved into distilled-deionized water (9 ml) and added to the stirring solution of the palladium salt. The solution went from a muddy orange mixture to a yellow clear solution in 30 minutes. The subsequent light yellow solution was reduced by the addition of sodium borohydride (1 % w/v, 1.10 ml of distilled-deionized water) in 0.10 ml portions. Immediately the color of the solution changed from light yellow to black. The dispersion was stirred vigorously for 1 hour. Upon standing, the dispersion remained stable with no observable settling of dispersion particles.

A dispersion was formulated with 5 ul of the palladium particles stabilized with the DMAP (4.8×10⁻⁷ mM/ul) and 5 ul of ethidium bromide (2.5×10⁻³ mM/ul). The dispersion was placed on a hybridized sensor for 10 minutes and then rinsed off with distilled water. The sensor was fitted in the electrical resistivity device described above. 10 ul of the nickel bath at 40° C. was placed over the sensor for 3.5 minutes. The sensor shorted out and microscopy showed thousands of nickel bridges spanning the electrodes indicating metallization of the nucleic acid polymer.

### EXAMPLE 4

A molecular scaffold was created by the partial hydrolysis of an ethyloxazoline polymer to create reactive secondary amine groups along the chain. A solution of polyethyloxazoline (15 g), isopropanol (62 g) and potassium hydroxide (4.8 g) was heated for 7 days at 85° C. The solution was diluted with an equal volume of water and the residual salts and alcohol were removed by dialysis. The water solution was then freeze dried to yield solid polymer with an estimated ratio of amine to amide repeat units of 1 to 5. An estimated lower ratio of 1 to 9 could be made with a shorter 4 day hydrolysis reaction.

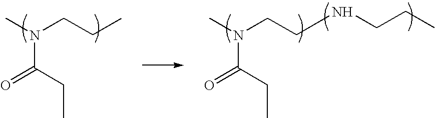

A reactive nucleic acid binding compound based on the napthalimide class of molecules was made as follows: A mixture of naphthalic anhydride (8g) and 3-(dimethylamino)-1-propylamine (4.1 g) in dimethylacetamide (50 g) was heated to 120 C to form a dark solution. The solvent was removed at reduced pressure, the mixture was dissolved in toluene (50 ml) and further distilled at atmospheric pressure to remove toluene-water azeotrope. The product was recrystallized from toluene to yield 4.9 g of 3-dimethyaminopropyl naphthalimide. Further reaction with a large excess of dibromohexane (4 g to 0.5 g ratio) in THF (4 g) at 60 C for 24 hours yielded a precipitate of the bromohexyl cationic salt (NI—C₃—N⁺—C₆Br).

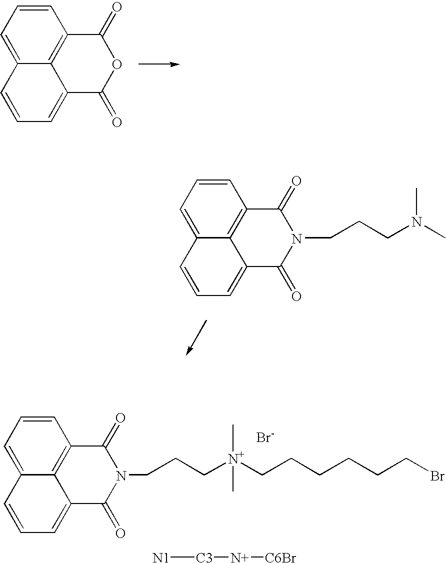

The cationic naphthalimide salt (NI—C₃—N⁺—C₆Br) was attached to the copolymer scaffold by alkylation of the amine groups with the terminal bromoalkyl group as follows: Copolymer (0.4 g) and NI—C₃—N⁺—C₆Br (0.105 g) in dimethylacetamide (1.2 g) were heated at 60° C. for 24 hours. The solution was then treated with an equivalent of triethylamine (0.02 g) and a three fold excess of ethyl isocyanate (0.048 g) in order to convert the remaining reactive amine groups to ethyl urea.

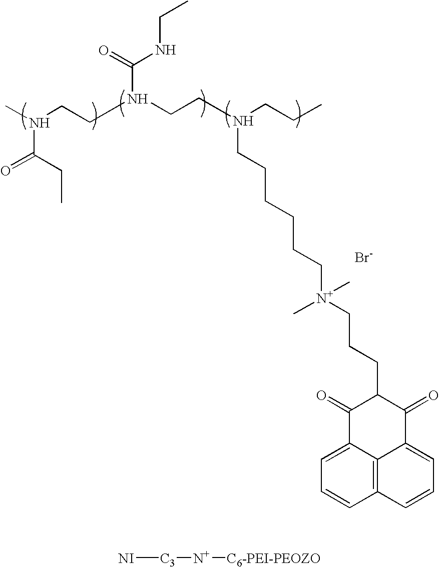

A palladium nanoparticle functionalized and stabilized with NI—C₃—N⁺—C₆-PEI-PEOZO was made as follows: A solution of 5 mg of sodium borohydride in 7.5 ml of dimethylacetamide was added drop wise over 30 minutes to a stirred solution of 47.4 mg of palladium acetate and 24mg of NI—C₃—N⁺—C₆-PEI-PEOZO in 15 ml of dimethylacetamide under a nitrogen atmosphere. After an additional 30 minutes of stirring in air, the black mixture was diluted with 20 ml of water and dialyzed for 48 hours to produce a stable aqueous dispersion that easily passed through a 0.2 u filter.

Nanoparticles functionalized with the C₃—N⁺—C₆-PEI-PEOZO oligomer were tested. The sensor chip containing nucleic acid polymer across the gold wires was first treated with a polyethyloxazoline solution (˜5% wt.) for 10 minutes and then treated with an equal volume of the test palladium nanoparticle (˜0.1% wt.) for 10 minutes. The sensor was then rinsed with water, fitted to the electrical resistivity measurement device and then immersed in the nickel metallization bath.

As shown in Table I, the palladium particles functionalized with a nucleic acid binding composition of this invention such as NI—C₃—N⁺—C₆-PEI-PEOZO led to an electrical connection. In addition, the NI—C₃—N⁺—C₆-PEI-PEOZO did not lead to non specific connection of wires with no nucleic acid present. Therefore, the advantage of the NI—C₃—N⁺—C₆-PEI-PEOZO functionalized palladium particle is demonstrated in Table I.

TABLE I
Time (min) to create electrical signal in nickel bath vs. DNA
level on chip and surface addenda on palladium particle.

| Particle Addenda | 50 ng of DNA | 0 ng of DNA |
|---|---|---|
| NI—C₃—N⁺—C₆-PEI-PEOZO | 1 to 4 min | >5 min |

### Preparation of Magnetite Clusters:

### EXAMPLE 5

A first solution of ferric chloride (0.8M), ferrous chloride (0.4M) and hydrochloric acid (0.4M) was mixed and 0.2 micron filtered. A second solution was prepared with 72 ml of ammonium hydroxide (30%) with water to make 1 liter.

1 ml of the ferric/ferrous chloride solution was added with stirring to 20 ml of the ammonium hydroxide solution. Stirring was continued for 15 seconds. The solution (in a 20 ml vial) was placed on a strong magnet and allowed to stand for 1 minute, after which all the product was pulled to the bottom of the vial. The clear supernatant liquid was decanted, replaced with water, mixed, and placed near the magnet. Again, the product was pulled to the bottom of the vial. This process was repeated three times to wash the product free from any residual ammonium and iron salts. The vial was then filled with 20 ml of water and ultra-sonicated for 5 minutes at 4 watts power. The suspension was then filtered through a 1 micron glass filter to give a stable suspension of magnetite particles that remain in suspension until pulled down by magnetic forces or centrifugation.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A nucleic acid binding substance comprising:
a paramagnetic nucleation nanoparticle containing paramagnetic elements;
a nucleic acid binding element capable of binding to a target nucleic acid polymer in communication with said catalytic nucleation nanoparticle.

2. The nucleic acid binding substance of claim 1 wherein said paramagnetic elements are cobalt, nickel or iron ions.

3. The nucleic acid binding substance of claim 1 wherein said paramagnetic nucleation nanoparticle further comprises a plurality of catalytic elements.

4. The nucleic acid binding substance of claim 3 wherein said catalytic elements are platinum, silver or palladium ions.

5. The nucleic acid binding substance of claim 3 wherein said catalytic nucleation nanoparticle contains a plurality of metallic atoms.

6. The nucleic acid binding substance of claim 1 wherein total binding energy between the nucleic acid binding element and the target nucleic acid polymer is at least 10 kJ/mole.

7. The nucleic acid binding substance of claim 1 wherein the nucleic acid binding element is selected from the group consisting of intercalators, minor groove binders, nucleic acids, proteins, cationic polymers and amine reactive groups.

8. The nucleic acid binding substance of claim 1 further comprising a stabilizer.

9. The nucleic acid binding substance of claim 8 wherein said nucleic acid binding element is a stabilizer.

10. The nucleic acid binding substance of claim 8 wherein said nucleic acid binding element is polyethylenimine.

11. The nucleic acid binding substance of claim 1 wherein said catalytic nucleation nanoparticle is substantially spherical with a diameter from 1 to 100 nm.

12. The nucleic acid binding substance of claim 1 further comprising a linker bound to said catalytic nucleation nanoparticle and in communication with said nucleic acid binding element.

13. The nucleic acid binding substance of claim 12 wherein the linker is selected from the group consisting of ligands, sequestering agents, amines, thiols, ionic binding agents, hydrophobic binding agents.

14. The nucleic acid binding substance of claim 1 further comprising a steric stabilizer associated with said catalytic nucleation nanoparticle and in communication with said nucleic acid binding element.

15. The nucleic acid binding substance of claim 14 wherein the steric stabilizer is selected from the group consisting of polyethylenimine, polyethyloxazoline and polyvinylpyrrolidone.

16. The nucleic acid binding substance of claim 1 further comprising a scaffold bound to said nucleic acid binding element and said catalytic nucleation nanoparticle.

17. The nucleic acid binding substance of claim 16 wherein said scaffold is bound to a plurality of nucleic acid binding elements.

18. A method for attaching a paramagnetic nucleation nanoparticle to a nucleic acid polymer comprising:
binding a nucleic acid binding element to a nucleic acid polymer;
providing a paramagnetic nucleation nanoparticle,
attaching the catalytic nucleation particle to the nucleic acid binding element.

19. The method for attaching a paramagnet nucleation nanoparticle to a nucleic acid polymer of claim 18 wherein said nucleic acid binding element further comprises a binding group capable of binding to said nucleic acid binding element.

20. A method for coating a nucleic acid polymer with a conductor comprising:
attaching a paramagnetic nucleation nanoparticle having a plurality of paramagnetic and catalytic elements to a nucleic acid polymer;
providing a developer solution that interacts with the plurality of catalytic elements causing conductive material to coat the target nucleic acid polymer.

21. The method of claim 20 wherein the catalytic elements are palladium, silver or platinum.

22. The method of claim 20 wherein the catalytic nucleation nanoparticle is attached to the nucleic acid polymer via a nucleic acid binding element.
